(19) United States
(12) Patent Application Publication
(10) Pub. No.: US 2009/0281530 A1
(43) Pub. Date: Nov. 12, 2009

(54) METHOD FOR TREATING AN ORGANIC MATERIAL

(75) Inventor: Georg Korn, Kleinmachnow (DE)

Correspondence Address: SCHMEISER, OLSEN & WATTS, 22 CENTURY HILL DRIVE, SUITE 302, LATHAM, NY, 12110, US

(73) Assignee: TECHNOLAS PERFECT VISION GMBH MESSERSCHMITTSTRASSE 1+3, MUNICH (DE)

(21) Appl. No.: 11/917,194

(22) PCT Filed: Jun. 12, 2006
(86) PCT No.: PCT/DE2006/001001

§ 371 Date: Jul. 9, 2008

(30) Foreign Application Priority Data
Jun. 13, 2005 (DE) 102005027355.6

(51) Int. Cl. A61F 9/008; A61B 18/20

(52) U.S. Cl. 606/5; 606/13

(57) ABSTRACT

The invention relates to a method for treating an organic material, in particular a biological material, in which the organic material is irradiated with laser light in the form of ultrashort pulses at a defined energy input, which pulses are adjusted with regard to a respective pulse length and pulse energy for the organic material such that an energy density of from around 100 mJ/cm² to around 100 J/cm² is created, wherein at least two pulses or a pulse sequence are irradiated consecutively onto a surface of the organic material and wherein a time interval between a preceding pulse/pulse sequence and a following pulse/pulse sequence is less than or equal to picoseconds, and the following pulse/pulse sequence hits the organic material again where the change effected by means of the preceding pulse/pulse sequence has taken place, with the result that a permanent change in the organic material is created.

The invention relates to a method for treating an organic material, in particular a biological material.

### PRIOR ART

Methods using ultrashort laser pulses for microstructuring materials known to date utilise the particular qualities of the rapid energy input of this radiation into the material to be machined. The pulses are typically shorter than the duration of the electron-phonon coupling of the material to be machined, which is typically less than 1 to 2 ps. That is, only a very small amount of energy is transferred to the surroundings of the localised interaction site during the action of the pulse. Destruction is therefore highly localised. Damage to the surroundings is less than with machining using longer pulses.

If longer pulses are used, it is possible for heat to flow into the surroundings of the irradiated site during the pulse. The spatial extent of the destroyed site can consequently be much greater than the radius of the effective laser beam. Investigations with CO₂ lasers have proved particularly drastic; these lasers have zones of influence up to 50 μm below the irradiated, ablated surface (see for example DE 101 25 206 B4).

Furthermore, a pronounced deterministic behaviour can be observed in the destruction process as a result of the high intensities. The cause of this is the reliable generation of initial free charge carriers (electrons) by multiphoton ionisation. Further destruction then occurs as a function of the pulse width either because of an avalanche process in the case of longer pulses or because of a direct multiphoton process in the case of shorter pulses with a pulse length of less than about 200 fs (cf. IEEE vol. 31. pp. 2241-2257 (1995); Appl. Phys. Lett. vol. 64, pp. 3071-3073 (1994)). In comparison to this, a statistical character can be seen with longer pulses with pulse lengths of more than about 10 ps of nanoseconds, as the pulse does not have the intensity to provide reproducibly the necessary initial charge carriers. The virtually free charge carriers are then distributed statistically in the material to be machined through easily ionisable defects. The non-deterministic destruction process for longer pulses follows from this.

It has furthermore been found that the destruction threshold (unit J/cm²) with longer pulses decreases proportionally to (τ)^{1/2} (τ—pulse width). For pulses in the region of or shorter than the electron-phonon coupling time, the curve falls much less steeply. Pulses with one ps or sub-ps widths require a much lower fluence than ns pulses for the destruction. Short single pulses in the sub-ps range are used for this reason in precision microstructuring for the machining of transparent and non-transparent materials, for surface machining and also for volume modification of materials (cf. U.S. Pat. No. 5,656,186).

The document DE 101 25 206 B4 discloses a method for direct microstructuring of materials by means of ultrashort single pulses or sequences of pulses with a defined energy input. In the known method, two laser pulses or pulse sequences that are shaped over time are directed consecutively towards the surface of the material to be machined. The interval between two consecutive pulses or pulse sequences is adjusted to be less than or equal to picoseconds, with the result that the following pulse hits the material to be machined again where the change effected by the first pulse has taken place. The energy and duration of the pulse are adjusted depending on the material to be machined. The known method is used to structure transparent or non-transparent materials in the micro range; concrete examples are solids such as graphite and fused silica. An identical method is described in the document EP 1 260 838 A2.

The optimum interval between the sub-ps pulses for improving machining results depends on the complex relationship between energy localisation and dissipation in the irradiated material. The use of specifically phase-modulated pulses or pulse sequences extends the options for machining solid materials by a possible increase in the intensity (irradiated energy per area) and a targeted “softening” of the material by the first pulse. The following pulses detect with a corresponding delay the changes in the state of the material brought about by the first pulse. This leads to the creation of improved structures with greater edge sharpness in the interior of the materials and on surfaces through the reduction of microcracks and stresses in the material.

### THE INVENTION

The object of the invention is to specify a method for treating an organic material, in particular a biological material, which method enables high-precision treatment, in particular for performing exact and additionally complex three-dimensional incisions or for creating cavities or changes in shape in transparent and non-transparent organic materials.

This object is achieved according to the invention by a method according to the independent claim 1. Advantageous developments of the invention form the subject matter of dependent subclaims.

According to the invention, a method is provided for treating an organic material, in particular a biological material, in which the organic material is irradiated with laser light in the form of ultrashort pulses at a defined energy input, which pulses are adjusted with regard to a respective pulse length and pulse energy for the organic material such that an energy density of from around 100 mJ/cm² to around 100 J/cm² is created, wherein at least two pulses or a pulse sequence are irradiated consecutively onto a surface of the organic material and wherein a time interval between a preceding pulse/pulse sequence and a following pulse/pulse sequence is less than or equal to picoseconds, and the following pulse/pulse sequence hits the organic material again where the change effected by means of the preceding pulse/pulse sequence has taken place, with the result that a permanent change in the organic material is created.

The time interval of the pulses of the consecutive pulses/pulse sequences and the duration of the individual pulses is consequently to be set to be shorter than the electron-phonon interaction time. Only in this case will the advantages of the method described here for improving the results of material treatment by means of shorter laser pulses have an effect.

An essential advantage of the invention consists in that for the organic material to be treated the radiation exposure for the area behind the irradiation site is reduced in the direction of propagation of the laser beam by means of the shaping over time of the irradiated light pulses and the targeted time sequence of the pulses/pulse sequences. In this way it is made possible for organic materials for changes to be carried out in a targeted fashion within the organic material, which are not restricted to the surface region, as is the case for other materials in the prior art (cf. DE 101 25 206 B4).

Furthermore, the advantage is produced that in comparison to known laser treatment methods, the accuracy of the treatment can be increased up to an accuracy of <1 μm, since the destruction is produced in a targeted way and restricted to the axis of the laser beam. This can be optimised by the interval between the pulses and the energy content as well as the shape over time of the pulses used.

In the method created a minimal energy density and intensity of the irradiated pulses/pulse sequences are guaranteed in order to achieve targeted deterministic destruction typical of the use of femtoseconds. In particular, the mean intensity in the interaction area of the radiation with the medium can be reduced with the described method. A short, intensive fs pulse with low energy generates the necessary initial density of free charge-carriers, a second delayed, longer sub-ps pulse irradiates the same, pretreated site. This second pulse can assume the function of actually treating the material, for example cutting or removing material in the interior of the material to be treated or on its surface.

A particular advantage in using the proposed method for treating biological materials, which can however also be advantageous for other organic materials, consists in that the radiation exposure can be optimised with respect to the intensity and energy density of the irradiated pulses/pulse sequences and can thereby be kept low in order to localise the destruction well and reduce boundary effects such as material stresses (in the volume around the destroyed site) which can arise due to excessively strong shock waves. In this way, the transparency of transparent materials is better retained after the treatment and the formation of scattering centres in the material is reduced. The homogeneity of the treated material is better retained.

The effect of this advantage is revealed in particular when the method is used for modifying cornea tissue in order to cut a “flap” within the context of a LASIK procedure (LASIK—Laser-Assisted in situ Keratomileusis) and for intrastromal correction of sight defects by means of the creation of specifically shaped cavities in order to correct myopia, hyperopia or various forms of astigmatism, and also when the method is used in wavefront-guided correction with reduction or elimination of higher-order aberrations. After such a treatment, a high contrast (visual acuity) as well as the necessary refraction of the eye should be produced. It is expected that photophobias (strong sensitivity to light) of the eye treated with fs pulses observed in some patients can be reduced or even completely eliminated. These are at least in some cases obviously the result of a combination of excessive radiation exposure of the retina by the cutting fs laser and the increased scattering of the treated stroma after the operation as well as the disruption of the healing process as a result of the change in the cornea with the effect of an intensive fs pulse.

An essential feature of irradiation with femtoseconds for the purpose of controlled precision destruction, for example cutting an incision or creating a cavity, is the time sequence of the process: At the start of the irradiation process, free charge carriers are initially created on the leading edge of the pulse as a result of multiphoton ionisation. The plasma produced does not yet have the critical density, however. As a result, part of the radiation energy used can pass through the interaction point. The biological tissue is in this case transparent and the radiation passing through impinges unchecked on the retina during an eye treatment. This can lead to dazzling of the photoreceptors or even to damage to the retina. It is therefore desirable to reduce these exposures on the retina to considerably below a critical value (1 μJ/cm²).

Measurements have shown that a reduction of the pulse width of 3 ps to 125 fs leads to a reduction in the transmitted energy during the plasma formation in water of around 20% for laser spot sizes between 7 μm and 10 μm (cf. Appl. Optics 36(22)5630-5640, 1997). This can be achieved in the proposed method in that a first short pulse with lower energy and high contrast ratio creates the critical plasma density. The intensity contrast of the first pulse should be about 10³ in order to create the interaction of the pulses with the material to be treated in a targeted manner in such a way that the advantages of laser material treatment with femtoseconds have an effect. The second, longer pulse that actually performs the treatment or the following parts of the specifically phase-modulated pulse are then completely absorbed in the dense plasma created, the laser energy passing through to the retina during the eye treatment is thereby reduced, and the local precision-controlled destruction of the cornea material is ensured. The intensity profile over time of the second pulse/pulse sequence can be optimally configured for example by means of adaptive control of the phase profile. An extended falling intensity profile over time, for example, is optimal for reducing the intensity exposure and for minimising the energy transmitted through the plasma. The energy actually necessary for the destruction and ablation is concentrated at the gradually tailing off end of the pulse.

Furthermore, it is important for the cornea incisions or the generation of cavities for intrastromal correction of sight defects to be carried out with a relatively short duration, for which reason the use of a highly repeating laser with energies in the region of a few μJ is preferred, for example about 0.1 to 1 μJ for the first pulse and about 1 to 5 μJ for the following pulse, in order to reduce the treatment time to short times, preferably much shorter than one minute. The use of a laser system with a high repeating frequency is also expedient for other applications, for comparable reasons.

Another observed phenomenon is the formation of “streaks”, which are patches of cloudiness in the stroma in the region of the interaction zone, for which a lateral extent of about 500 nm has been observed, and which are visible up to about 100 μm in both directions along the laser beam in the corneal stroma treated with fs pulses (see Heisterkamp, Appl. Phys. B74, 419-425). This is the consequence of self-focussing of the intensive fs pulses in front of the intended site of interaction, as the peaks of the fs pulses can be above a critical value. The “streaks” can lead to cloudiness in the cornea. Reducing the mean intensity, as is possible in the method proposed here, and selecting a suitable focussing angle reduce these undesired exposure effects or eliminate them altogether. This results in a clear cornea after treatment with the fs pulses. A reduction in the number of cases of photophobia following fs treatment can be expected.

With a wavefront-guided ablation, improvements with respect to correction of aberrations are achieved by means of increasing the precision of the incision and improved repositioning of the flap following treatment with the method presented here. In particular in LASIK procedures, the mechanical deformations occurring as a result of the incision with the microkeratome have until now had a severe, often non-reproducible influence on the residual aberrations produced in the eye after treatment. An improvement in the accuracies of the fs laser incision and in the achievable repositioning accuracy of the flap leads to the desired mean square wavefront deformation in the order of magnitude below λ/10 (RMS—“root mean square”) or λ/14, as a result of which the theoretical diffraction-limited resolution behaviour is approached. Because of the increase in the accuracy of the process, this will make a further improvement in wavefront-guided ablation with ns UV pulses possible.

Although individual advantages of the invention have been explained above in connection with treating biological tissue in the eye, the respective advantages also reveal their positive effects in a corresponding manner with other organic materials, for example in connection with operations on the auditory canal or in the treatment of tooth material.

### EXEMPLARY EMBODIMENTS OF THE INVENTION

The invention will be explained in detail below using exemplary embodiments and with reference to figures, in which:

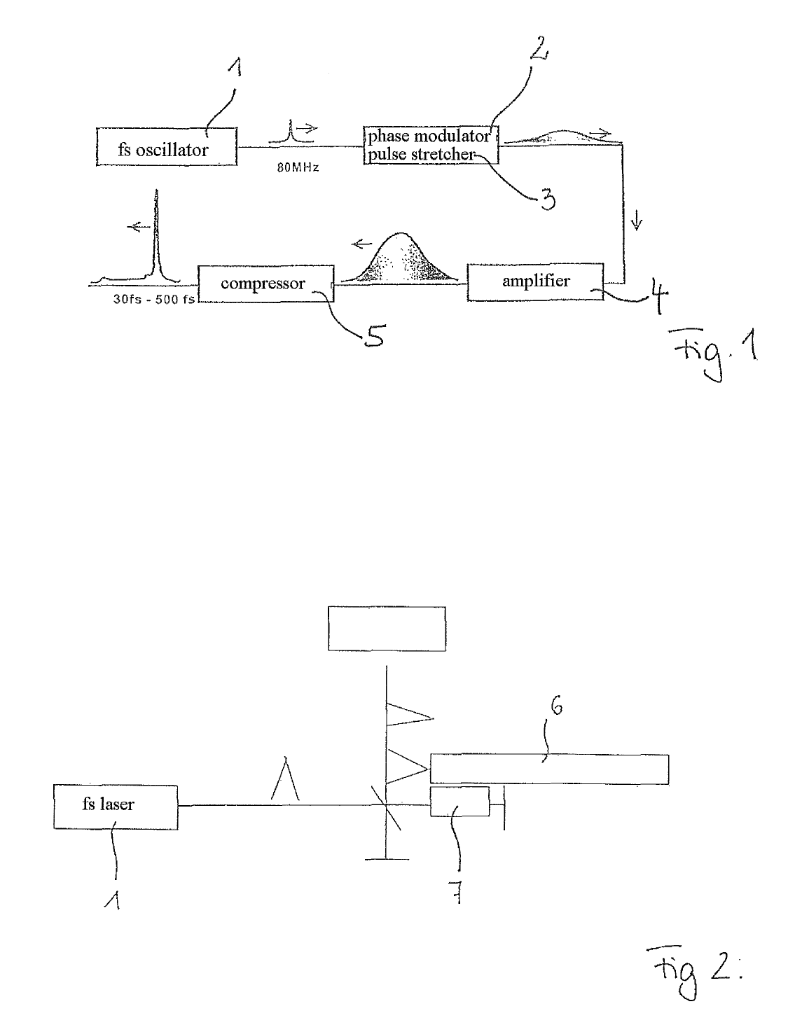

FIG. 1 shows a schematic representation of a laser system for use in the treatment of an organic material with ultrashort light pulses, and

FIG. 2 shows a schematic representation of an arrangement for generating pulses, using a Michelson interferometer to generate an additional phase modulation in the laser system according to FIG. 1.

FIG. 1 shows a schematic representation of a CPA laser system (CPA—chirped pulse amplification), which is used to generate ultrashort pulses for treating an organic material. The time profiles and the energy of the generated pulses/pulse sequences, which can be specifically phase-modulated, can be adjusted for treating the respective organic material. Modulation is generally achieved by adapting the oscillator level.

Pulses generated by an fs oscillator 1 having a repeating frequency of about 80 to 100 MHz are passed to a phase modulator 2. The pulse treated in this way then passes to a pulse stretcher 3, which impresses a linear “chirp” in addition to the existing phase modulation. The pulses then amplified in an amplifier 4 are then converted into high-energy fs pulses in a compressor 5. The pulses/pulse sequences generated in this way, which are phase-modulated on account of the non-compensated, additionally impressed phase modulation, are then used for treating the organic material, for example in the context of making an incision for a LASIK procedure or of correcting sight defects by making intrastromal changes in the eye.

An additional/separate phase modulation is also provided. In conventional CPA laser systems in the range above 100 fs, the linear “chirp” of the pulse stretcher 3 is mainly eliminated by the compressor 5. The pulse formed in this way has virtually its original pulse length after amplification.

In an extension of the laser system according to FIG. 1, an arrangement according to FIG. 2, for example, can be used to provide an optimised double pulse for the treatment, which pulse fulfils the above-mentioned criteria for a treatment of the organic material that is as gentle as possible. In FIG. 2, the same reference symbols as in FIG. 1 are used for the same features.

The pulse is sent from the fs oscillator 1 through a phase modulator 2 configured as a Michelson interferometer. An adjustable dispersive element 7 is additionally present in an optical arm 6, which element impresses one of the generated pulses with a mainly 2nd order adjustable dispersion, in addition to a delay. The two pulses, which can be delayed with respect to one another in an adjustable manner, are then stretched in the pulse stretcher 3 to a value between about 10 ps and 500 ps.

After amplification, the compressor 5 is adjusted in such a way that the pulse width of only the first pulse has a value of about 50 to 500 fs. The following pulse can have an adjustable width between about 100 fs and 3 ps as a function of the dispersion additionally impressed in the Michelson interferometer 2, with an adjustable time delay between the amplified pulses. The treatment result is optimised through the selection of the time delay and the additionally introduced dispersion. Phase modulation methods can be achieved by changing the phase delay in a targeted manner with different wavelengths, for example in a “zero dispersion stretcher” or by means of acousto-optical methods. The result of the material treatment can be optimally configured by means of computers with the aid of a feedback loop (A. M. Weiner, “Femtosecond Pulse Shaping Using Spatial Light Modulators”, Rev. Sci Instrum. 71, pp. 1929-1960, 2000.)

The essential thing is that the described CPA system stretches the pulse in terms of time in the stretcher for the purpose of amplification (cf. U.S. RE37,585). This extended pulse can then be brought to high energies in the amplifier system, without non-linear effects becoming noticeable during the amplification. After amplification, the pulse is then compressed back to its original length. In contrast to this, the pulse or pulses in this document have an additional phase modulation at the stretcher, which cannot or should not be compensated by the compressor and thereby leads to the formation of double pulses, pulse sequences or pulses specifically shaped over time, which have variable pulse lengths or intervals and allow the treatment process to be optimised in the above-described manner.

Particularly suitable for carrying out the method are directly diode-pumped femtosecond laser systems having a central wavelength in the region of 1 μm. Yb-doped materials with a low Stoke's shift, which can be pumped efficiently and cost-effectively with low-power diodes, should be pointed out. The bandwidth of these materials allows the generation and amplification of pulses to below 100 fs. The oscillators can be configured by means of SESAMs (SESAM—“Semiconductor Saturable Absorber Mirror”), which has improved the stability and reliability of these systems substantially. Wavelengths around 1 μm are especially well suited for treating the eye, as the cornea has good transparency and the sensitivity of the photoreceptors of the retina is greatly reduced in comparison to near infrared (Ti:sapphire lasers). Phase modulation methods can be generally used for femtosecond systems.

The energy density that can be achieved is not increased. The intensity should be reduced as a whole by being divided into multiple pulses, pulse sequences or specifically phase-modulated pulses, in order to produce a material treatment method that is very precise, less damaging and as minimally invasive as possible.

The features of the invention disclosed in the above description, the claims and the drawings can be of significance both individually and in any combination for implementing the invention in its various embodiments.

1. Method for treating an organic material, in particular a biological material, in which the organic material is irradiated with laser light in the form of ultrashort pulses at a defined energy input, which pulses are adjusted with regard to a respective pulse length and pulse energy for the organic material such that an energy density of from around 100 mJ/cm² to around 100 J/cm² is created, wherein at least two pulses or a pulse sequence are irradiated consecutively onto a surface of the organic material and wherein a time interval between a preceding pulse/pulse sequence and a following pulse/pulse sequence is less than or equal to picoseconds, and the following pulse/pulse sequence hits the organic material again where the change effected by means of the preceding pulse/pulse sequence has taken place, with the result that a permanent change in the organic material is created.

2. Method according to claim 1, characterised in that organic material is treated by means of ablation or destruction.

3. Method according to claim 1, characterised in that the surface of the organic material is structured.

4. Method according to claim 1, characterised in that the surface of the organic material is cut.

5. Method according to claim 1, characterised in that one or more incisions are made in the interior of the organic material.

6. Method according to claim 4, characterised in that an incision is made according to a predefined three-dimensional model.

7. Method according to claim 1, characterised in that the interior of the organic material is structured.

8. Method according to claim 1, characterised in that organic material in an eye is treated.

9. Method according to claim 8, characterised in that a LASIK (Laser-Assisted In Situ Keratomileusis) incision is made in a cornea.

10. Method according to claim 9, characterised in that a wavefront-guided ablation is carried out during the LASIK incision.

11. Method according to claim 9, characterised in that the LASIK incision is made in a lamellar fashion according to a predefined three-dimensional model.

12. Method according to claim 8, characterised in that main and auxiliary incisions are made in a sclera and/or cornea for a cataract operation.

13. Method according to claim 8, characterised in that scleral and/or corneal incisions are made for a vitrectomy (pars plana) and incisions are made in a vitreous cavity and in a retina.

14. Method according to claim 8, characterised in that asymmetrical incisions are made in three-dimensional form for keratoplasty.

15. Method according to claim 8, characterised in that tissue is incised in the chamber angle (in the context of a goniotomy) for glaucoma surgery, or fistulising incisions are made through other tissue sections.

16. Method according to claim 8, characterised in that lens tissue is modified for controlling presbyopia and in cataract surgery is loosened during planned removal of tissue.

17. Method according to claim 8, characterised in that the muscle is cut during a squint operation with the objective of shortening or extending it.

18. Method according to claim 8, characterised in that closed tear ducts are widened or opened.

19. Method according to claim 8, characterised in that loosening cuts are made in the cornea in the horizontal, vertical and other three-dimensional directions in order to correct refraction anomalies.

20. Method according to claim 1, characterised in that cavities and/or channels are formed in the organic material.

21. Method according to claim 1, characterised in that the ultrashort pulses are generated in a short pulse laser using a pulse-forming method.

22. Method according to claim 21, characterised in that a pulse-forming, phase-modulating method is used in a CPA laser in order to amplify the ultrashort pulses.

23. Method according to claim 1, characterised in that a high-energy, “cavity-dumped” oscillator is used.

24. Method according to claim 1, characterised in that an active element of a laser consisting of directly diode-pumped Yb-doped materials such as Yb glass, KY(WO₄)₂, KGd(WO₄)₂, Sc₂O₃, CaF₂ or Y₂O₃ ceramic material is used in an oscillator and/or an amplifier, with laser beam wavelengths in the infrared spectral range of around 1.0 μm to around 1.2 μm.

25. Method according to claim 1, characterised in that a fibre laser doped with rare earth metals is used as a pulse-generating laser.

26. Method according to claim 25, characterised in that a fibre amplifier is used, which has a double sheath structure.
